(19) United States
(12) Patent Application Publication
(10) Pub. No.: US 2004/0221853 A1
(43) Pub. Date: Nov. 11, 2004

(54) Ultrasonic placement and monitoring of a tube within the body

(75) Inventor: Michael Miller, Minneapolis, MN (US)

Correspondence Address: Glenn M. Seager, CROMPTON, SEAGER & TUFTE, LLC, 1221 Nicollet Avenue, Suite 800, Minneapolis, MN, 55403-2420, US

(73) Assignee: PLASIATEK, LLC

(21) Appl. No.: 10/730,690

(22) Filed: Dec. 8, 2003

Related U.S. Application Data
(60) Provisional application No. 60/468,665, filed on May 8, 2003.

(51) Int. Cl. A61M 16/00

(52) U.S. Cl. 128/207.14; 128/205.23; 128/200.26; 600/433

(57) ABSTRACT

Apparatus and methods for ultrasonically placing and monitoring a tube within the body are disclosed. A tubular apparatus in accordance with an exemplary embodiment may include a tubular member having a proximal section, a distal section, and at least one inflatable member on the tubular member in fluid communication with an external fluid source containing an acoustically transmissive material. The inflatable member may be configured to expand at least in part within a hollow body cavity or conduit to permit the tubular apparatus to be ultrasonically visualized using an ultrasound unit located outside of the patient's body.

This application relates to Provisional Application No. 60/468,665, filed May 8, 2003.

### FIELD OF THE INVENTION

The present invention relates generally to the field of medical devices. More specifically, the present invention pertains to apparatus and methods for ultrasonic placement and monitoring of a tube within the body.

### BACKGROUND OF THE INVENTION

A number of medical procedures require the insertion of a tube, catheter, cannula, or other similar device into the body. Such devices are used, for example, in the fields of anesthesiology, cardiology, endoscopy, urology, laparoscopy, and vascular therapy to deliver fluids such as oxygen and anesthetics to targeted regions within the body. In the field of anesthesiology and critical care, for example, it may be necessary to deliver air/oxygen to the anesthetized patient using an endotracheal tube (ETT). Such tubes are routine used in the clinical, ICU, emergency room, and pre-hospital settings to restore and maintain an adequate airway to the lungs, to prevent the inspiration of forced air into the stomach via the esophagus tube, and to protect against the aspiration of gastric contents into the lungs.

In a typical endotracheal intubation procedure, the distal end of the ETT is inserted through either the mouth or nose and is advanced into the trachea, generally at a location midway between the vocal folds and the carina. An inflatable balloon cuff located at or near the distal end of the ETT can be inflated to secure the ETT within the trachea, providing and air seal that allows the caregiver to completely control the flow of air provided to the lungs using an external ventilator unit, and that can be used to prevent the aspiration of gastric contents into the lungs.

The placement and monitoring of the ETT within the body remains a significant obstacle in endotracheal intubation procedures. Malpositioning may result when the ETT is inadvertently placed into the esophagus tube, causing air to be injected into the stomach instead of the trachea. Endobronchial intubation caused by over-extending the ETT past the carina and into one of the right or left primary bronchi may also exacerbate the intubation process, resulting in the ventilation of only one of the lungs. In certain circumstances, the lung that is being improperly ventilated may become hyperventilated due to the higher concentrations of inspired oxygen, causing barotraumas and hypotension. Atelectasis of the unventilated lung may also result from the improper insertion of the ETT into the bronchi.

Movement of the ETT once placed within the trachea may further exacerbate the intubation process. Flexion or extension of the patient's neck can change the desired positioning of the ETT, in some cases resulting in extubation from the trachea. Such changes in head position are common with normal patient movement in the ICU, emergency room, and pre-hospital settings. In addition, mucus, blood, or other biological materials may also result in the movement or blockage of the ETT, requiring further action by the caregiver to ensure proper ventilation of the patient. In any of these scenarios, the lack of proper ventilation within the patient may lead to cardiac arrest or irreversible central nervous system damage within a relatively short period of time.

The efficacy of endotracheal intubation procedure depends in part on the ability of the caregiver to quickly and accurately determine the positioning of the ETT within the body. Most intubation devices and methods rely on the ability to visualize the opening to the trachea and place the ETT by direct vision, typically with the aid of another instrument such as a fiber optic laryngoscope. Anatomical variations from patient to patient can, however, render direct visualization of the trachea opening difficult and in some cases impossible. This is particularly so during critical care and emergency procedures where the positioning of the patient's head or the presence of blood or saliva may exacerbate direct visualization. Post placement movement or blockage of the ETT may also be undetectable using direct visualization techniques, rendering this method ineffectual for monitoring of the ETT once inserted into the trachea.

To address these problems, various devices and techniques have been developed to aid in the proper placement and monitoring of the ETT within the body. Known techniques include, for example, chest radiography, stethoscopic evaluation of airway breath and epigastric sounds, visualization of the trachea and carina using a fiber optic bronchoscope, visualization of the vocal cords or trachea by video methods, pulse oximetry, carbon dioxide (CO₂) measurements, colorimetric end tidal CO₂ (ETCO₂) measurements, electromagnetic sensing, suction techniques, and the observation of symmetric bilateral movements of the chest wall during ventilation. A review of the various types of instruments utilized in the art is provided in U.S. Pat. No. 5,785,051 to Lipscher et al., which is incorporated herein by reference in its entirety.

More recent designs in the art have focused on ultrasonic techniques to monitor the placement of tubes within the body. Such designs generally include an ultrasonic transducer mounted directly on the tube that can be used to transmit acoustic waves to a receiver located either on another portion of the tubular member, or to an external receiver located outside of the patient's body. In several prior art designs, the ability to ultrasonically visualize the tube is often dependent on the distance between the transducer and receiver, rendering such techniques prone to error in those applications where the distance is great, or where acoustical obstructions such as bone or air are present. In endotracheal intubation procedures, for example, a weak or nonexistent signal received from the transducer may falsely indicate that an esophageal intubation has occurred, requiring the caregiver to remove the ETT from the patient's body and reattempt the intubation process. Moreover, air located in the trachea, larynx, pharynx, and esophagus may impair ultrasonic imaging of these structures, affecting the ability of the caregiver to assess whether any contraindications to tracheal intubation exist.

While several prior art designs permit the caregiver to confirm the position of the tube once it has been placed in the body, such devices are not capable of ultrasonic placement and monitoring of the tube in real-time. Abnormalities in the airway and variations from patient to patient may render many ultrasonic techniques unsatisfactory for use. As such, there is a need in the art to provide real-time ultrasonic placement and monitoring of a tube within the body.

### SUMMARY OF THE INVENTION

The present invention pertains to apparatus and methods for ultrasonic placement and monitoring of a tube within the body. A tubular apparatus in accordance with an exemplary embodiment of the present invention may include a tubular member having a proximal section, a distal section, and a ventilation lumen disposed therebetween. A first inflatable member disposed about the tubular member may be inflated with air, saline or other suitable fluid to secure the tubular apparatus within a hollow body cavity or conduit. In endotracheal intubation procedures, for example, the first inflatable member can be configured to radially expand and secure the tubular apparatus within the trachea, providing an air seal that allows the caregiver to ventilate the patient using the ventilation lumen.

A second inflatable member disposed about the tubular member distally of the first inflatable member may be used to ultrasonically place and monitor the tubular apparatus within the body. The second inflatable member may be coupled to an external fluid source that contains an acoustically transmissive material having an acoustical impedance matched with that of the surrounding anatomy. The second inflatable member can be configured to expand symmetrically or asymmetrically when inflated, allowing the inflatable member to engage all or a portion of the cavity or conduit interior.

An ultrasound unit including one or more ultrasonic transducers located outside of the patient may be configured to direct an ultrasonic beam through the skin and into the second inflatable member. The ultrasound unit may include an ultrasonic monitor capable of ultrasonically imaging the tubular apparatus and surrounding anatomy from the backscatter of acoustic waves through the second inflatable member. In certain embodiments, for example, the ultrasound unit may include an ultrasonic monitor capable of ultrasonically imaging fluid flow within the second inflatable member using Doppler imaging techniques. Auscultatory devices capable of audibly confirming the presence of fluid motion within the second inflatable member may also be employed to determine the positioning of the tubular apparatus within the body.

### BRIEF DESCRIPTION OF THE DRAWINGS

FIG. 1 is a perspective view of a tubular apparatus in accordance with an exemplary embodiment of the present invention;

FIG. 2 is a cross-sectional view of the tubular apparatus of FIG. 1 along line 2-2;

FIG. 3 is an enlarged view of the distal section of the tubular apparatus of FIG. 1;

FIG. 4 is an enlarged view of the distal portion of a tubular apparatus in accordance with another exemplary embodiment of the present invention;

FIG. 5 is an enlarged view of the distal portion of a tubular apparatus in accordance with another exemplary embodiment of the present invention;

FIG. 6 is a cross-sectional view showing the airway of a human patient;

FIG. 7 is a cross-sectional view of the human patient of FIG. 6, showing the distal section of the tubular apparatus of FIG. 1 inserted into the patient and advanced to a position near the vocal cords;

FIG. 8 is a cross-sectional view of the human patient of FIG. 6, showing the distal section of the tubular apparatus of FIG. 1 advanced to a position near the epiglottis and opening of the trachea;

FIG. 9 is a cross-sectional view of the human patient of FIG. 6, showing the leader cuff balloon in an inflated position near the epiglottis and opening of the trachea;

FIG. 10 is an enlarged view of the distal section of the tubular apparatus of FIG. 1, showing the injection of fluid into the leader balloon cuff;

FIG. 11 is a cross-sectional view of the human patient of FIG. 6, showing the distal section of the tubular apparatus secured to the wall of the trachea; and

FIG. 12 is a cross-sectional view of the human patient of FIG. 6, showing the leader balloon cuff in an inflated position within the trachea.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

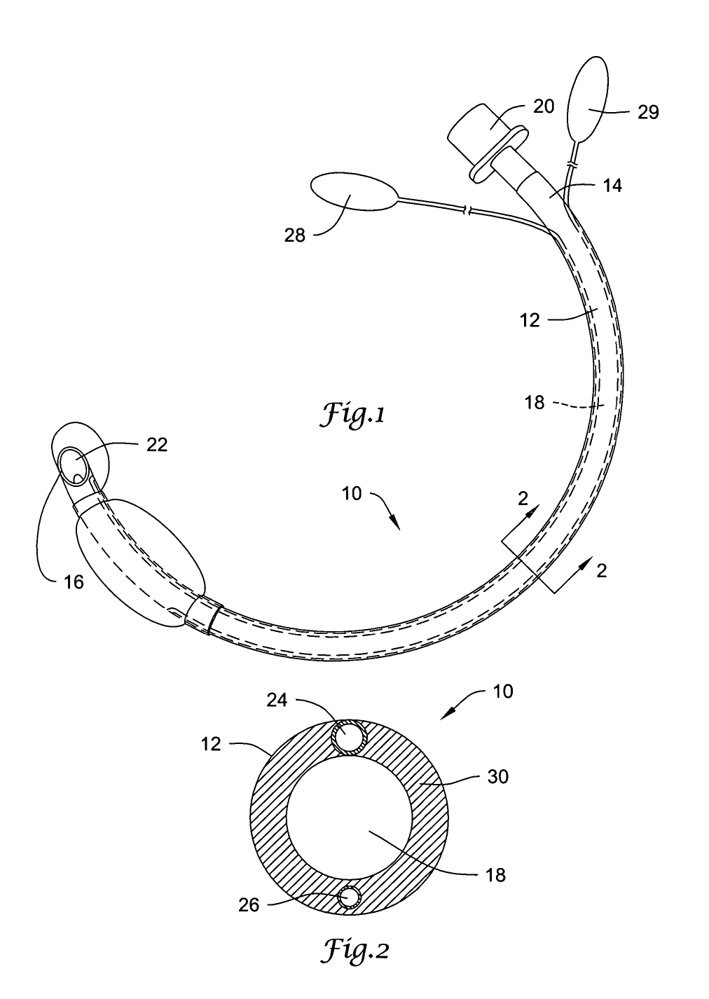

FIG. 1 is a perspective view of a tubular apparatus 10 in accordance with an exemplary embodiment of the present invention. Tubular apparatus 10, illustratively an endotracheal tube (ETT) suitable for endotracheal intubation procedures, includes a tubular member 12 having a proximal section 14, a distal section 16, and a ventilation lumen 18 disposed at least in part therethrough. The proximal section 14 of the tubular member 12 may be coupled to a ventilation hub 20, which fluidly connects the ventilation lumen 18 to an external ventilation unit (not shown) that can be activated to provide air to and from the patient's lungs via a distal opening 22 in the tubular member 12.

The tubular member 12 may comprise a suitably flexible material to permit the tubular apparatus 10 to be easily inserted into the patient's airway. The tubular member 12 may also be provided with sufficient rigidity along its length to withstand buckling and transmit torque as it is inserted into the body. In certain embodiments, the tubular member 12 may have a substantially curved shape along its length that approximates the contour of the patient's airway, allowing the tubular apparatus 10 to follow a pre-guided path through the anterior portion of the larynx/pharynx and into the trachea. Other configurations such as a substantially straight shape may also be implemented, if desired.

The tubular member 12 may have a length of approximately 9 to 15 inches and an outer diameter of about 0.7 cm to 1.1 cm, which is suitable for most adult orotracheal intubation procedures. The dimensions of the tubular member 12 may, however vary for use in other applications, as necessary. In intubations for small infants, for example, the length and cross-sectional area of the tubular member 12 can be scaled down to accommodate the relatively small size of the undeveloped infant trachea, which is typically about 4 cm in length and 0.5 cm in diameter. Moreover, where orotracheal intubation is unfeasible or contraindicated (e.g. in the case of a suspected cervical spine injury), the tubular member 12 can be appropriately sized to permit alternative intubation techniques such as nasotracheal intubation or cricothyrotomy. The dimensions of the tubular member 12 can also be altered to permit the device to be used in other fields such as veterinary medicine, if desired.

FIG. 2 is a cross-sectional view of the tubular apparatus 10 of FIG. 1 along line 2-2. As shown in FIG. 2, tubular apparatus 10 includes a main ventilation lumen 18 that can be used to provide air, anesthetics, or other vital fluids to targeted regions of the body. In certain embodiments, the ventilation lumen 18 may have an inner diameter of about 0.3 cm to 1.3 cm, and more specifically 0.6 cm to 0.8 cm, which is sufficient for most orotracheal and nasotracheal intubation procedures. It should be understood, however, that the inner diameter of the ventilation lumen 18 may vary based on factors such as the size of the endotracheal tube employed, or the type of procedure performed.

While a single ventilation lumen 18 is specifically depicted in FIG. 2, it should be understood that the tubular apparatus 10 can include a plurality of ventilation lumens disposed therethrough. In certain intubation procedures, for example, it may be desirable to inspire air into only one of the lungs using, for example, a tubular apparatus having two separate ventilation lumens disposed along its length. When inserted at an appropriate location within the patient's airway (e.g. below the carina), the double lumen tubular apparatus can be used, for example, to ventilate only one of the patient's lungs, or to provide a differential level of ventilation to each of the lungs.

As can be further seen in FIG. 2, the tubular apparatus 10 may further define a first inflation lumen 24 and second inflation lumen 26, which can be utilized to deliver fluid to and from several inflatable balloon members disposed about the tubular member 12. The first inflation lumen 24 extends along at least part of the length of the tubular member 12, and is fluidly coupled to an external fluid reservoir 28 (FIG. 1) such as an elastomeric bulb, syringe mechanism, or the like that can be used to inject and/or aspirate fluids through the first inflation lumen 24. The second inflation lumen 26 similarly extends along at least part of the length of the tubular member 12, and is fluidly coupled to a second fluid source 29 that can be used to inject and/or aspirate fluids through the second inflation lumen 26.

In the illustrative embodiment depicted in FIG. 2, the first and second inflation lumens 24,26 are each disposed within the tubular wall 30 on opposite sides from each other. The first and second inflation lumens 24,26 may be formed either integral with the tubular member 12 using, for example, a molding process, or as separate components that are later secured to the tubular member 12 by a suitable attachment process such as adhesive or heat bonding. In an alternative embodiment (not shown), the first and/or second inflation lumens 24,26 can be configured to extend along the inside or outside of the tubular member 12, either on opposite sides of the tube wall 30, or in some other desired arrangement.

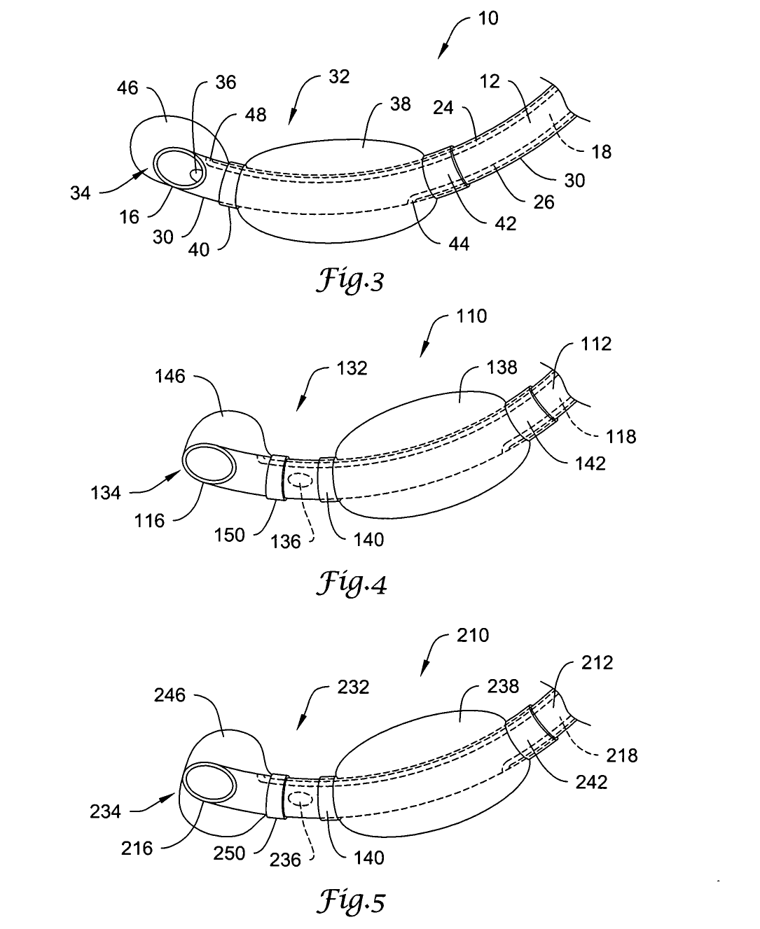

FIG. 3 is an enlarged view of a distal section 32 of the tubular apparatus 10 of FIG. 1. As can be seen in FIG. 3, the distal section 16 of the tubular member 12 may have a beveled shape, forming a tip 34 on the posterior wall of the tubular member 12. The tip 34 may comprise a material that is sufficiently soft and flexible to prevent trauma to the body as the tubular apparatus 10 is advanced within the patient's body. In certain embodiments, a Murphy eye 36 located on the posterior wall of the tubular member 12 may also be provided to prevent complete blockage of the tubular apparatus 10 in the event the distal opening 22 becomes partially or totally occluded.

An inflatable main balloon cuff 38 disposed about the distal section 32 of the tubular member 12 may be used to secure the tubular apparatus 10 to the interior wall of the trachea during intubation. The main balloon cuff 38 can be secured to the outer surface of the tubular member 12 using a number of sleeves 40,42 that can be bonded to the tubular member 12 by adhesive, heat bonding, or other suitable bonding technique. The main balloon cuff 38 can be configured to inflate when fluid (e.g. air, saline solution, etc.) in the second inflation lumen 26 is injected through an opening 44 in the tube wall 30 and into the interior of the main balloon cuff 38.

As shown in an expanded position in FIG. 3, the main balloon cuff 38 can be configured to radially expand in a symmetrical manner about the tubular member 12 to permit the tubular apparatus 10 to engage both the anterior and posterior portions of the tracheal wall, thereby securing the tubular apparatus 10 within the trachea. When fully inflated, the main balloon cuff 38 can be used to occlude the airway surrounding the tubular apparatus 10, allowing the physician to regulate the patient's respiration using an external ventilation unit (not shown) fluidly coupled to the ventilation lumen 18, and to protect against the aspiration of gastric contents or other foreign matter into the lungs.

In a further aspect of the present invention, tubular apparatus 10 may include a leader balloon cuff 46 that can be utilized to ultrasonically place and monitor the tubular apparatus 10 within the body. In the exemplary embodiment of FIG. 3, the leader balloon cuff 46 is shown attached to the tubular member 12 at a location distal to the main balloon cuff 38 using one of the sleeves 40. The leader balloon cuff 46 can be injected with fluid via opening 48, which is in fluid communication with the first inflation lumen 24 and the external fluid reservoir 28.

To permit rapid inflation and deflation of the leader balloon cuff 46, the size of the first inflation lumen 24 can made relatively large to reduce the effects of head loss as fluid is delivered along the length of the tubular apparatus 10. As can be seen by reference to FIG. 2, for example, the first inflation lumen 24 may have an inner diameter slightly greater than the inner diameter of the second inflation lumen 26. This enlargement reduces the effects of head loss within the lumen, allowing the leader balloon cuff 46 to be quickly inflated and deflated within the body. Other design factors such as the length of the first inflation lumen 24 and the type of inflation fluid employed may also be varied to affect the rate at which the leader balloon cuff 46 can be inflated and deflated.

The leader balloon cuff 46 can be configured to inflate in an asymmetric or symmetric manner about the tubular member 12. As illustrated in an inflated position in FIG. 3, for example, the leader balloon cuff 46 can be configured to expand asymmetrically to permit the leader balloon cuff 46 to engage the anterior surface of the trachea without fully occluding the airway. The leader balloon cuff 46 can be configured to expand in part in a direction away from the longitudinal axis of the tubular member 12. In certain embodiments, the leader balloon cuff 46 can also be configured to expand in part beyond the distal end of the tubular member. In use, the leader balloon cuff 46 can be inflated with an acoustically transmissive fluid that can be utilized in conjunction with one or more ultrasonic transducers and an optional ultrasonic monitor located outside of the patient's body to visualize the distal section 32 of the tubular member 12 in vivo.

FIG. 4 is an enlarged view of the distal section 132 of a tubular apparatus 110 in accordance with another exemplary embodiment of the present invention. Tubular apparatus 110 includes a tubular member 112 equipped with a distal section 116 having a beveled shape that forms a tip 134 on the posterior wall of the tubular member 112, similar to that described above with respect to FIG. 3. A Murphy eye 138 located on the posterior wall of the tubular member 112 between the main balloon cuff 138 and leader balloon cuff 146 can be provided to prevent complete blockage of the tubular apparatus 110 in the event the distal opening 122 becomes partially or totally occluded with mucus, blood, or other debris.

In the exemplary embodiment of FIG. 4, the main balloon cuff 138 and leader balloon cuff 146 are disposed further apart from each other, and are attached to the tubular member 112 at different locations than the tubular apparatus 10 described above. The main balloon cuff 138 can be secured to the outer surface of the tubular member 112 using a number of sleeves 140,142. A third sleeve 150 separate from the sleeves 140,142 used to secure the main balloon cuff 138 can be used to secure the leader balloon cuff 146 to the tubular member 112. In use, the main balloon cuff 138 and leader balloon cuff 146 may function in a manner similar to that described above with respect to FIG. 3.

FIG. 5 is an enlarged view of the distal section 232 of a tubular apparatus 210 in accordance with another exemplary embodiment of the present invention. Tubular apparatus 210 includes a tubular member 212 equipped with a distal section 216 having a beveled shape that forms a tip 134 on the posterior wall of the tubular member 212. A main balloon cuff 238 can be secured to the outer surface of the tubular member 212 using a number of sleeves 240,242. A leader balloon cuff 246 disposed distally of the main balloon cuff 238 is further shown attached to the outer surface of the tubular member 212 with a third sleeve 250, similar to that described above in FIG. 4.

As can be seen in FIG. 5, the leader balloon cuff 238 can be configured to expand symmetrically about the circumference of the tubular member 212 when inflated. This symmetric expansion allows the caregiver to visualize the leader balloon cuff 238 irrespective of the orientation of the tubular apparatus 2120 within the body. While the illustrative leader balloon cuff 238 of FIG. 5 is shown having a substantially curved shape with an outer profile similar to that of the expanded main balloon cuff 238, it should be understood that the leader balloon cuff 246 could assume any number of sizes and/or shapes, as desired.

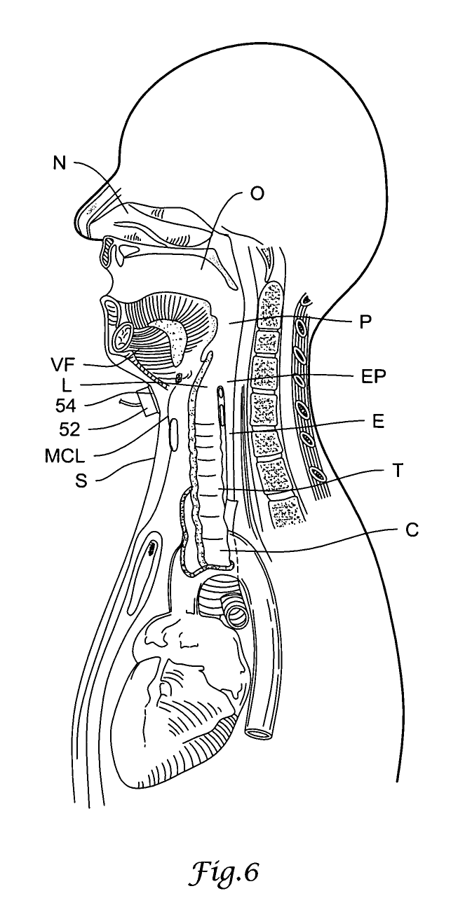

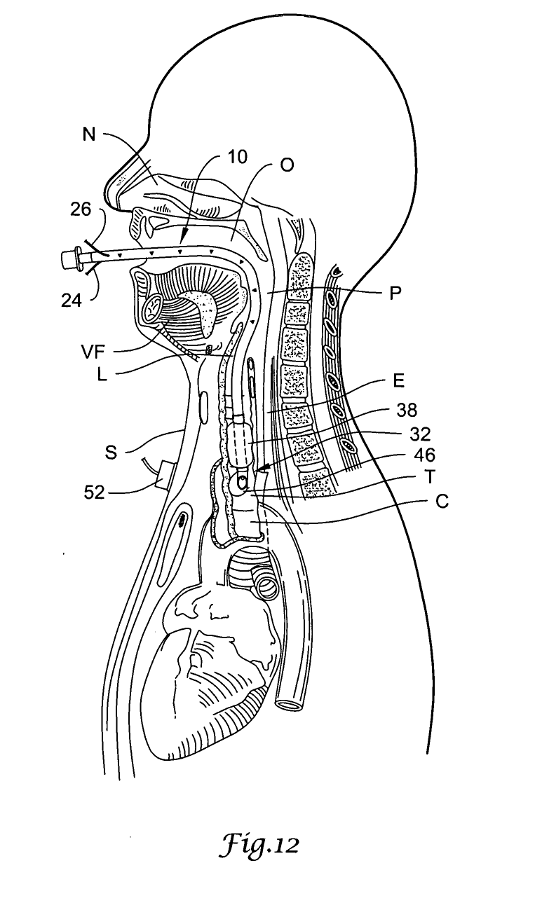

Referring now to FIGS. 6-12, an exemplary method of ultrasonically placing and monitoring a tube within the body will now be described in the context of an orotracheal intubation procedure using the tubular apparatus 10 described above. While specific reference is made to endotracheal intubation procedures, it should be understood that the methods described herein could be used in a number of other medical procedures to place and monitor tubes within the body. The methods described herein, for example may be used in vascular interventional procedures to place and monitor tubes used in vascular brachytherapy, angioplasty, stent placement, vascular catheter placement, or the like. Other medical fields including, for example, endoscopy, cardiology, urology, laparoscopy, obstetrics, neurology, radiology, and emergency medicine may also benefit from the methods described herein.

FIG. 6 is a cross-sectional view showing the airway of a human patient prior to insertion of the tubular apparatus 10 within the body. As illustrated in FIG. 6, the upper or cranial portion of the trachea T is characterized by the larynx L and pharynx P, which contain the vocal folds VF and epiglottis EP. The lower or caudal portion of the trachea T, in turn, contains a first bifurcation known as the carina C, which leads to the bronchi of the lungs. The adult trachea T extends approximately 9 to 15 cm in length, and is surrounded by various cartilage and ligaments, including the thyroid cartilage, the cricoid cartilage, and the middle cricothyroid ligament MCL.

In preparation for the intubation procedure, the caregiver places an ultrasonic transducer 52 about the anterior surface S of the patient's neck. The ultrasonic transducer 52 may include one or more piezoelectric elements made from lead zirconate titanate (PZT) or other suitable material responsive to frequencies above 1 MHz. A gel pad 54 and/or ultrasonic gel having an acoustic impedance similar to that of the skin may be placed between the ultrasonic transducer 52 and the anterior surface S of the neck to reduce reflection loss, thus improving transmission of the ultrasound into the adjacent tissue. An optional neck strap or other suitable fastening mechanism can also be used to secure the ultrasonic transducer 52 and gel pad 54 to the anterior surface S of the neck.

The ultrasonic transducer 52 can be connected to an external ultrasonic monitor that can be used to visualize the larynx L, pharynx P, trachea T, vocal folds VF as well as other surrounding anatomy prior to insertion of the tubular apparatus 10 within the body. Such initial step may be performed, for example, to assess whether any abnormalities exist that may make the intubation process difficult, or in determining whether alternative airway management methods are indicated. In certain circumstances, for instance, an initial ultrasonic scan of the patient's airway may lead to the discovery of an obstruction in the upper portion of the trachea, indicating that an alternative method such as a cricothyrotomy may be necessary.

Ultrasonic imaging of the larynx L, pharynx P, vocal folds VF, trachea T, and surrounding anatomy can be accomplished using any number of suitable ultrasonic imaging techniques in the art, including, for example, A mode imaging, B mode imaging, C mode imaging, M mode imaging, Doppler or Duplex imaging, and/or Power Doppler imaging. In certain embodiments, the ultrasonic transducer and monitor may be provided as a single, portable unit that can be used in a pre-hospital setting such as at an accident site or in an ambulance. Such portable ultrasonic devices are commercially available from SonoSite, Inc. of Brothell, Washington.

Once the caregiver has determined that tracheal intubation is appropriate, a metal stylet or other stiffening member may be temporarily inserted into the ventilation lumen 18 of the tubular apparatus 10 to provide rigidity for the intubation process. Furthermore, in preparation for insertion, the caregiver can place the ultrasonic transducer 52 on the patient's neck. In certain embodiments, the ultrasonic transducer 52 can be placed against the anterior surface S of the patient's neck and oriented in a slight upward position towards the vocal folds VF, allowing the caregiver to obtain an initial visual confirmation of the tubular apparatus 10 once inserted into the body, if desired.

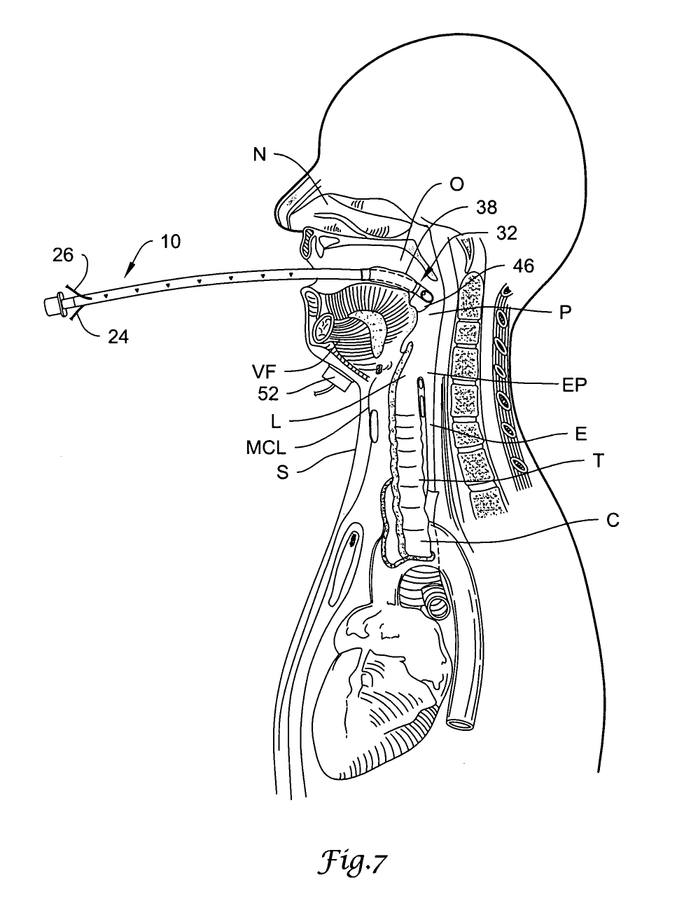

With the ultrasonic transducer 52 positioned on the patient's neck, the caregiver next inserts the tubular apparatus 10 and accompanying metal stylet into the patient, either through the mouth or the nose in accordance with standard practice in the art. In an orotracheal intubation approach illustrated in FIG. 7, for example, the distal section 32 of the tubular apparatus 10 can be inserted through the patient's oral cavity O, and then advanced to the region of the vocal folds VF. During this process, both inflatable balloon cuffs 38,46 can be maintained in a deflated position to facilitate passage of the tubular apparatus 10 through the airway.

While an orotracheal intubation approach is specifically shown in FIG. 7, it should be understood that the tubular apparatus 10 could also inserted through the patient's nasal cavity N if a nasotracheal intubation approach is indicated. In such approach, the distal section 32 of the tubular apparatus 10 can be inserted through the patient's nasal cavity N, and then advanced to the vocal folds VF. As with an orotracheal approach, both balloon cuffs 38,46 can be maintained in a deflated position to facilitate passage through the airway.

To provide confirmation that the tubular apparatus 10 has been inserted through the vocal folds VF, the caregiver can inflate and then subsequently deflate the leader balloon cuff 46 one or more times. When the leader balloon cuff 46 is inflated within the region of the vocal folds VF, ultrasonic waves transmitted from the ultrasound transducer 52 are allowed to pass into the leader balloon cuff 46 and reflect against the distal section 32 of the tubular apparatus 10. As is discussed in greater detail below, these reflected waves are then transmitted back through the leader balloon cuff 46 and surrounding anatomy to a receiver outside of the patient's body, allowing the caregiver to ultrasonically determine the placement of the tubular apparatus 10 within the body.

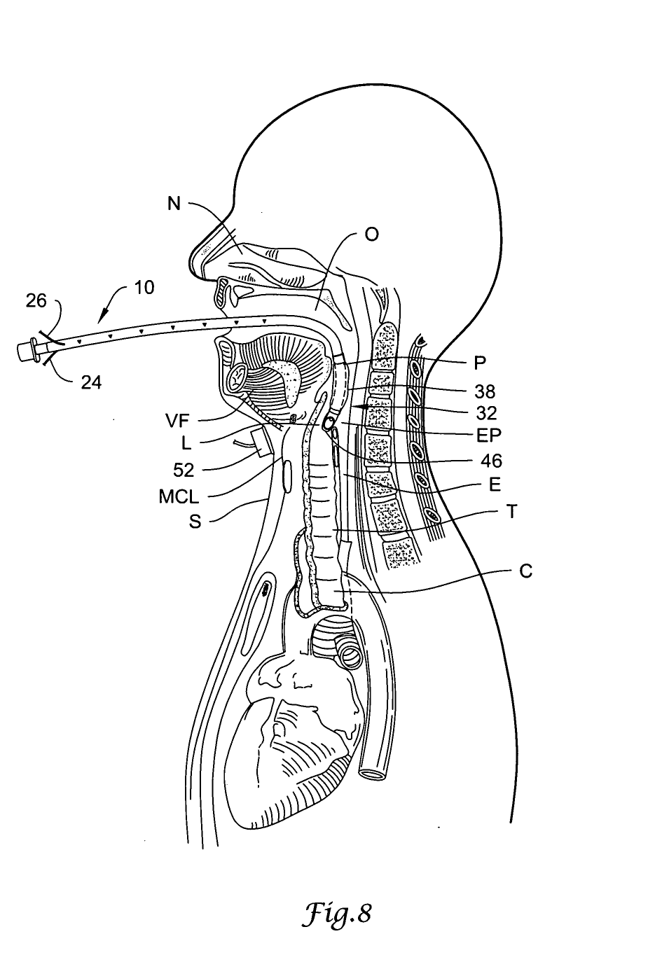

Once confirmation that the distal section 32 of the tubular apparatus 10 has been inserted and advanced to a position near the vocal folds VF, the caregiver next advances the tubular apparatus 10 to a second position within the body at or near the epiglottis EP and opening of the trachea T, as shown in FIG. 8. In preparation for this step, the caregiver may position the ultrasonic transducer 52 on the anterior surface S of the neck at a location adjacent or cephalad to the patient's middle cricothyroid ligament MCL, orienting the ultrasonic transducer 52 in a direction such that the ultrasonic beam passes through the middle cricothyroid ligament MCL and into the larynx/pharynx. The middle cricothyroid ligament MCL is a section of tissue located anterior to the trachea T between the thyroid cartilage and the cricoid cartilage. At this location, ultrasonic waves transmitted by the ultrasonic transducer 52 are easily passed through the tissue and soft ligament due to their impedance characteristics relative to the surrounding cartilage. The amount of reflection loss is dependent in part on the acoustic impedance (Z) between successive layers, which is determined based on the general formula:

Z=ρc

wherein ρ is the density of the material and c is the speed of sound in the material.

In general, the greater the difference in the acoustic impedance (Z) between two successive layers, the greater the amount of reflection loss that will occur as the ultrasonic wave passes through each layer. For waves passing normally from a first material (Z₁) to a second material (Z₂), the ratio of reflected intensity I_{R} to the initial intensity I_{O} may be determined in accordance with the general formula:

I_{R}/I_{O}=(Z₂−Z₁)²/(Z₂+Z₁)²

Because the acoustic impedance (Z) of the middle cricothyroid ligament MCL is similar to that of the surrounding tissue, ultrasonic waves tend to pass easily through the ligament and into the larynx L. As a result, ultrasonic imaging at this location tends to produce greater resolution and less interference (i.e. speckle) than at other locations. Other factors such as the consistency of depth from the patient's skin to the middle cricothyroid ligament MCL, the relatively fixed tracheal diameter in adult patients, the location of the esophagus E relative to the trachea T, and the ease of locating the ligament relative to external features on the anterior surface S of the patient's neck also suggest placement of the ultrasonic transducer 52 at this location.

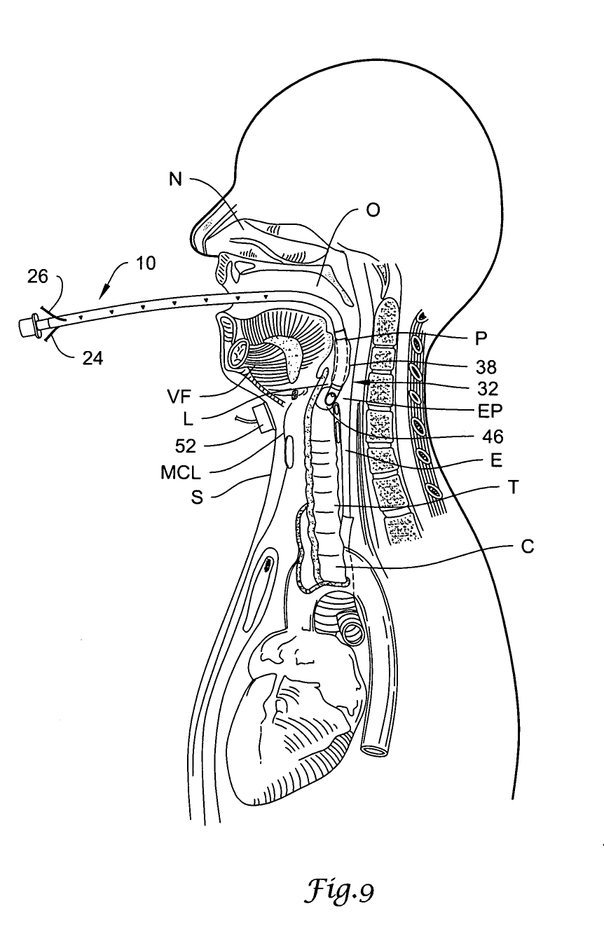

To guide the tubular apparatus 10 into the trachea T, the physician, while holding the tubular apparatus 10 in one hand, engages the external fluid reservoir 28 forcing the leader balloon cuff 46 to inflate within the body, as shown in FIG. 9. In those embodiments employing an elastomeric bulb for the external fluid reservoir 28, the caregiver can squeeze or release the elastomeric bulb using the ring and little fingers, causing the inflatable balloon cuff 46 to selectively inflate and deflate.

As shown in an inflated position in FIG. 9, the leader balloon cuff 46 is configured to expand asymmetrically or symmetrically about the tubular member 12 against the anterior surface of the larynx/pharynx, displacing the air within the larynx/pharynx with fluid located in the leader balloon cuff 46. With the air displaced at this region, the ultrasonic wave transmitted from the ultrasonic transducer 52 is allowed to pass into the interior of the leader balloon cuff 46 and backscatter against the outer surface of the tubular member 12, producing an image on the ultrasonic monitor that can be utilized to determine the positioning of the tubular apparatus 10 within the body.

To increase transmission of ultrasound waves through the leader balloon cuff 46, the fluid used to inflate the leader balloon cuff 46 may include an acoustically transmissive fluid having an acoustic impedance (Z) similar to that of surrounding anatomy. In certain embodiments, for example, the fluid may comprise a balanced saline solution having a density (ρ) similar to the adjacent tissue and ligament within the larynx/pharynx, allowing the ultrasonic beam to pass through the interior of the leader balloon cuff 46 and backscatter against the outer diameter of the tubular member 12. Other materials such as foam, gel, or other pseudo-fluidic materials having a certain desired acoustical impedance characteristic may also be employed, if desired.

Imaging of the ultrasound can be further improved by the selection of materials used in the formation of the leader balloon cuff 46. In certain embodiments, for example, the leader balloon cuff 46 can be formed from an echo-opaque material having an acoustic impedance (Z) selected for its ability to permit ultrasound waves to pass from the surrounding tissue into the interior of the leader balloon cuff 46 without significant reflection and/or attenuation. While balanced saline solution may be preferred in certain applications, other fluid, mixtures, emulsions, or combinations thereof may be implemented to facilitate ultrasonic imaging of the tubular apparatus 10 within the body, if desired.

Using ultrasonic imaging techniques, proper placement of the tubular apparatus 10 at the anterior portion of the larynx/pharynx can be confirmed in real-time using the ultrasonic monitor. Because air is a poor conductor of ultrasonic waves due to its relatively low characteristic impedance, the incorrect insertion of the tubular apparatus 10 into the esophagus E instead of the trachea T will not produce an image on the ultrasonic monitor, informing the caregiver that an esophageal intubation may have occurred. In certain embodiments, the ultrasonic monitor can be configured to provide an audible and/or visual alarm indicating that the tubular apparatus 10 has been improperly placed in the esophagus or at some other undesired location. The tubular apparatus 10 may then be repositioned and again confirmed by inflating the leader balloon cuff 46 until detected by the ultrasound.

In certain embodiments, the presence of fluid flow through the leader balloon cuff 46 may be visualized using Doppler imaging techniques. Generally, when the ultrasound beam transmitted from the ultrasonic transducer is scattered by a target having a component of velocity along the direction of the propagation, the frequency of the scattered ultrasound is shifted by the Doppler effect. If θ is defined as the angle between the target motion and the ultrasound beam, then:

v=−f_{D}c/(2fcosθ)

wherein v is the speed of the target and ƒ_{D} is the difference between the frequencies of the ultrasound transmitted from the transducer and backscattered along the ultrasonic beam, provided that v<<c.

Using this basic principal, a measure of the speed (v) of the fluid injected into or aspirated from the interior of the leader balloon cuff 46 can be calculated and, if desired, displayed on an ultrasonic monitor. In certain embodiments, for example, a color Doppler flow imaging monitor capable of simultaneously superimposing a color Doppler image on a gray-scale B-mode image, M-mode image, Power Doppler image, or other type image can be used to visualize fluid movement caused by fluid injected into and/or aspirated from the leader balloon cuff 46. Typically, a color such as red is assigned to represent fluid flow towards the ultrasonic transducer 52, whereas a color such as blue is assigned to represent flow away from the ultrasonic transducer 52. The magnitude of the velocity may be indicated by different shades of the color; the lighter the color typically representing a higher velocity.

Because the difference frequency (f_{D}) between the incident wave and the backscattered wave typically exists within the audible frequency range (i.e. 20 to 20 kHz), the existence of fluid motion within the leader balloon cuff 46 can also be heard using an auscultatory device such as the Doptone®. Such auscultatory devices can be utilized, for example, to confirm the existence of the distal section 32 of the tubular apparatus 10 at a particular location within the body without using ultrasonic imaging techniques. This may be useful in certain settings such as the pre-hospital setting where an ultrasonic monitor may not be readily available.

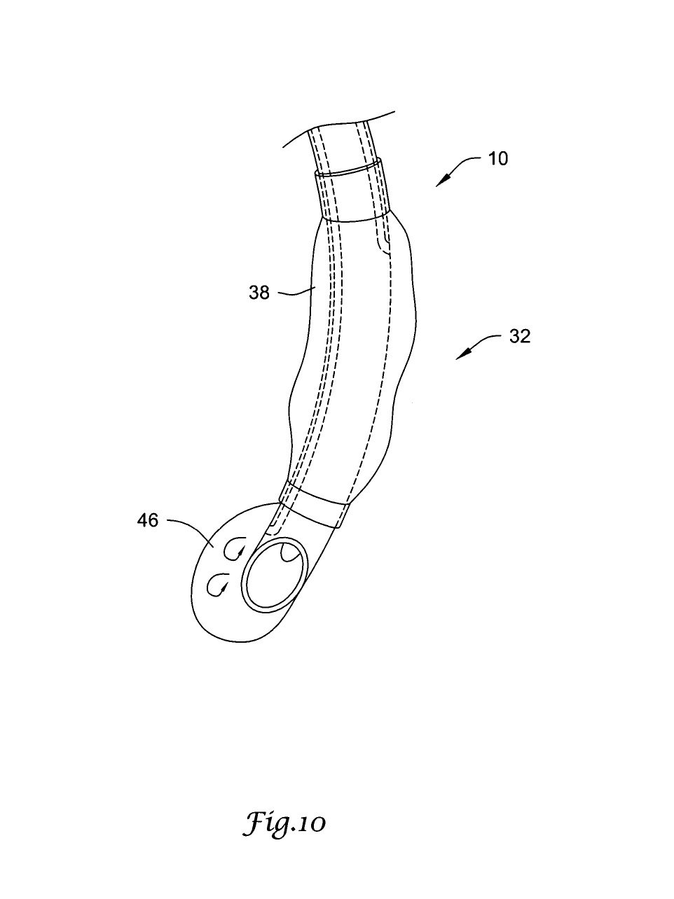

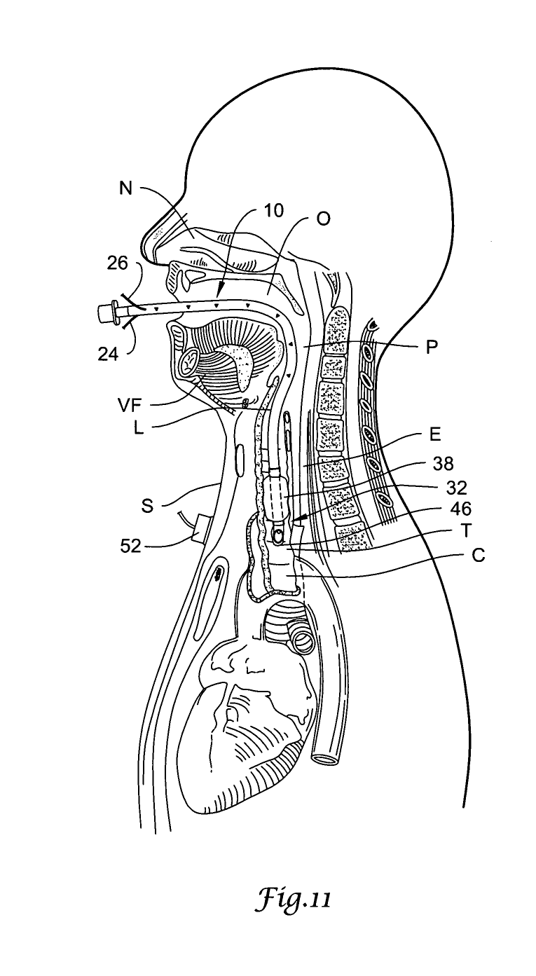

FIG. 10 is an enlarged view of the distal section 32 of the tubular apparatus 10 of FIG. 1, showing the injection of an acoustically transmissive fluid into the interior of the leader balloon cuff 46. As shown in FIG. 10, the injection and/or aspiration of fluid within the leader balloon cuff 46 creates eddy currents within the interior of the leader balloon cuff 46, indicated generally by the curved arrows depicted in FIG. 10. When imaged with a color Doppler ultrasonic monitor, for example, these eddy currents produce red or blue flow lines on the monitor that can be used to distinguish the distal section 32 of the tubular apparatus 10 from the surrounding anatomy. The fluid movement within the leader balloon cuff 46 can be distinguished on the ultrasonic monitor from other fluid-filled conduits in the body, which typically travel through the body without significant rotation.

Once the caregiver has determined that the tubular apparatus 10 is properly positioned along the anterior portion of the larynx/pharynx at or near the epiglottis EP, the tubular apparatus 10 can then advanced into the trachea T guided by the location of the Doppler image as well as the gray-scale image created by the tubular apparatus 10 and surrounding anatomy. Once tracheal intubation has been confirmed, the tubular apparatus 10 is then further advanced into the trachea T by imaging the leader cuff balloon 26 with the trachea T by inflating and deflating the leader balloon cuff 46 with fluid.

As the tubular apparatus 12 is being advanced into the trachea T, the ultrasonic transducer 52 can be configured to follow the movement of the leader balloon cuff 46 along the anterior surface S of the patient's neck to prevent misalignment of the ultrasonic beam and leader balloon cuff 46. Alignment of the ultrasonic transducer 52 may be accomplished manually, by physically moving the location of the ultrasonic transducer 52 on the patient's neck, or automatically with the aid of a tracking device (not shown) that automatically adjusts the location of the ultrasonic transducer 52 along the neck. In certain embodiments, the ultrasonic transducer 52 may include a vertical array of transducer elements that can be selectively activated on the surface S of the patient's neck to track the movement of the tubular apparatus 10 within the trachea T.

FIG. 11 is a cross-sectional view showing the tubular apparatus 10 secured to the wall of the trachea T. As shown in FIG. 11, once the tubular apparatus 10 has been advanced to a location at or near the midpoint of the trachea T, the main balloon cuff 38 can be inflated with air, saline or other suitable fluid, causing the main balloon cuff 38 to expand and engage the interior surface of the trachea T. With the trachea T fully occluded by the main balloon cuff 38, the caregiver can then ventilate the patient using the ventilation lumen 18 and attached ventilation unit, consistent with standard practice.

To periodically verify the positioning of the tubular apparatus 10 within the trachea T, the leader balloon cuff 46 can be periodically inflated at any time to produce a real-time image of the apparatus 10 on the ultrasonic monitor. Using this image, the caregiver can then visually confirm proper placement of the tubular apparatus 10 within the trachea T. In certain embodiments, the ultrasonic monitor can be configured to provide a visual and/or audible alert informing the caregiver that the tubular apparatus 10 has moved from its pre-set location within the trachea T. For example, if extubation of the tubular apparatus 10 has occurred, the ultrasonic monitor may sound an alarm informing the caregiver that reinsertion is necessary.

While the specific apparatus and methods discussed herein use a single balloon to ultrasonically monitor the location of the tubular apparatus within the body, other embodiments have been envisioned in which multiple balloons containing an acoustically transmissive material may be employed. In one alternative embodiment, for example, a number of inflatable members disposed at other locations along the length of the tubular member may be used to visualize other sections of the apparatus within the body. In another alternative embodiment, the tubular apparatus may include a single inflatable member that serves both the function of securing and visualizing the apparatus within the body.

The methods described herein can be utilized either alone or in conjunction with other devices and/or methods used in the art. Other visualization methods such as laryngoscopy may be used in addition to the methods described herein, as appropriate, to aid in the placement and/or monitoring of the tubular apparatus within the body.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention as described in the appended claims.

What is claimed is:

1. A tubular apparatus insertable within the body, comprising:
a tubular member having a proximal section, a distal section, and a lumen therethrough;
a first inflatable member coupled to the distal section of the tubular member, said first inflatable member being in fluid communication with a first fluid source;
a second inflatable member coupled to the distal section of the tubular member, said second inflatable member being in fluid communication with a second fluid source containing an acoustically transmissive material; and
an extracorporeal ultrasound unit for ultrasonically placing and monitoring the tubular apparatus within the body.

2. The tubular apparatus of claim 1, wherein the distal section of said tubular member includes a beveled tip.

3. The tubular apparatus of claim 1, wherein the tubular member has a curved shape.

4. The tubular apparatus of claim 1, further comprising an opening disposed through the wall of the tubular member.

5. The tubular apparatus of claim 4, wherein said opening is located between the first and second inflatable members.

6. The tubular apparatus of claim 1, further comprising a ventilation hub connected to the proximal section of said tubular member, said ventilation hub being in fluid communication with the ventilation lumen and an external ventilation unit.

7. The tubular apparatus of claim 1, wherein the second inflatable member is coupled to the tubular member at a location distal to the first inflatable member.

8. The tubular apparatus of claim 1, wherein the first inflatable member is configured to radially expand in a symmetrical manner about the tubular member.

9. The tubular apparatus of claim 1, wherein the second inflatable member is configured to expand in an asymmetrical manner about the tubular member.

10. The tubular apparatus of claim 1, wherein the second inflatable member is configured to expand in a symmetrical manner about the tubular member.

11. The tubular apparatus of claim 1, wherein the acoustically transmissive material comprises a balanced saline solution.

12. The tubular apparatus of claim 1, wherein the ultrasound unit includes at least one ultrasonic transducer.

13. The tubular apparatus of claim 12, further comprising an ultrasonic monitor capable of ultrasonically imaging the second inflatable member within the body.

14. The tubular apparatus of claim 13, wherein said ultrasonic monitor is adapted to ultrasonically image fluid flow within the second inflatable member using Doppler imaging.

15. The tubular apparatus of claim 1, wherein said tubular member is an endotracheal tube.

16. An endotracheal tubular apparatus insertable within the body, comprising:
a tubular member having a proximal section, a distal section, and a ventilation lumen therethrough;
a first inflatable member coupled to the distal section of the tubular member, said first inflatable member being in fluid communication with a first fluid source;
a second inflatable member coupled to the distal section of the tubular member, said second inflatable member being in fluid communication with a second fluid source containing an acoustically transmissive material; and
an extracorporeal ultrasound unit for ultrasonically placing and monitoring the tubular apparatus within the body.

17. The endotracheal tubular apparatus of claim 16, wherein the distal section of said tubular member includes a beveled tip.

18. The endotracheal tubular apparatus of claim 16, wherein the tubular member has a curved shape.

19. The endotracheal tubular apparatus of claim 16, further comprising a Murphy eye disposed through the wall of the tubular member.

20. The endotracheal tubular apparatus of claim 19, wherein said Murphy eye is located between the first and second inflatable members.

21. The endotracheal tubular apparatus of claim 16, further comprising a ventilation hub connected to the proximal section of the tubular member, said ventilation hub being in fluid communication with the ventilation lumen and an external ventilation unit.

22. The endotracheal tubular apparatus of claim 16, wherein the second inflatable member is coupled to the tubular member at a location distal to the first inflatable member.

23. The endotracheal tubular apparatus of claim 16, wherein the first inflatable member is configured to radially expand in a symmetrical manner about the tubular member.

24. The endotracheal tubular apparatus of claim 16, wherein the second inflatable member is configured to expand in an asymmetrical manner about the tubular member.

25. The endotracheal tubular apparatus of claim 16, wherein the second inflatable member is configure to expand in a symmetrical manner about the tubular member.

26. The endotracheal tubular apparatus of claim 16, wherein the acoustically transmissive material comprises a balanced saline solution.

27. The endotracheal tubular apparatus of claim 16, wherein the ultrasound unit includes at least one ultrasonic transducer.

28. The endotracheal tubular apparatus of claim 27, further comprising an ultrasonic monitor capable of ultrasonically imaging the second inflatable member within the body.

29. The endotracheal tubular apparatus of claim 28, wherein said ultrasonic monitor is adapted to ultrasonically image fluid flow within the second inflatable member using Doppler imaging.

30. An endotracheal tubular apparatus insertable within the body, comprising:
a tubular member having a proximal section, a distal section, and a ventilation lumen therethrough;
at least one inflatable member coupled to the distal section of the tubular member, said at least inflatable member being in fluid communication with a fluid source containing an acoustically transmissive material; and
an extracorporeal ultrasound unit for ultrasonically placing and monitoring the tubular apparatus within the body, said ultrasound unit including at least one ultrasonic transducer configured to direct an ultrasonic beam through the skin and into said at least one inflatable member.

31. A method of ultrasonically placing and monitoring an endotracheal tubular apparatus within a patient's airway, comprising the steps of:
providing at least one ultrasonic transducer on the anterior surface of the patient's neck;
providing an endotracheal tubular apparatus including a ventilation lumen, a main balloon cuff in fluid communication with a first external fluid source, and a leader balloon cuff in fluid communication with a second external fluid source;
inserting at least a portion of the tubular apparatus into the patient's oral or nasal cavity and advancing the tubular apparatus to a position at or near the patient's epiglottis;
inflating the leader balloon cuff with an acoustically transmissive material, causing the leader balloon cuff to expand against the anterior portion of the patient's larynx/pharynx;
ultrasonically determining the position of the tubular apparatus within the body;
advancing the tubular apparatus to a position within the trachea; and
expanding the main balloon cuff to provide an air seal within the trachea.

32. The method of claim 31, further comprising the step of ultrasonically imaging the patient's airway prior to the step of inserting the tubular apparatus into the body.

33. The method of claim 31, further comprising the step of ultrasonically confirming the position of the tubular apparatus at or near the vocal folds prior to said step of advancing the tubular apparatus to a position at or near the patient's epiglottis.

34. The method of claim 31, wherein the step of ultrasonically determining the position of the tubular apparatus within the body comprises the steps of:
directing an ultrasonic beam through the surface of the skin and into the leader balloon cuff; and
viewing the resulting image on an ultrasonic monitor.

35. The method of claim 34, wherein the ultrasonic beam is passed through the patient's middle cricothyroid ligament.

36. The method of claim 34, wherein the ultrasonic monitor is adapted to ultrasonically image fluid flow within the leader balloon cuff using Doppler imaging.

37. The method of claim 31, further comprising the step of ultrasonically confirming proper placement of the tubular apparatus within the trachea after said step of advancing the tubular apparatus to a position within the trachea.

38. The method of claim 37, wherein said step of ultrasonically confirming proper placement of the tubular apparatus comprises the steps of:
periodically injecting the leader balloon cuff with the acoustically transmissive material, causing the leader balloon cuff to expand against the anterior portion of the patient's trachea; and
ultrasonically imaging the location of the tubular apparatus within the trachea.

39. The method of claim 31, further comprising the steps of ventilating the patient using an external ventilating unit operatively coupled to the ventilation lumen.

40. A method of ultrasonically placing and monitoring an endotracheal tubular apparatus within a patient's airway, comprising the steps of:
providing at least one ultrasonic transducer on the anterior surface of the patient's neck;
providing an endotracheal tubular apparatus including a ventilation lumen, a main balloon cuff in fluid communication with a first external fluid source, and a leader balloon cuff in fluid communication with a second external fluid source;
inserting at least a portion of the tubular apparatus into the patient's oral or nasal cavity and advancing the tubular apparatus to a position at or near the patient's epiglottis;
inflating the leader balloon cuff with an acoustically transmissive material, causing the leader balloon cuff to expand against the anterior portion of the patient's larynx/pharynx;
ultrasonically imaging the location of the tubular apparatus within the body;
advancing the tubular apparatus to a position within the trachea;
expanding the main balloon cuff to provide an air seal within the trachea; and
periodically inflating the leader balloon cuff with the acoustically transmissive material to ultrasonically confirm proper placement of the tubular apparatus within the trachea.

41. The method of claim 40, further comprising the step of ultrasonically imaging the patient's airway prior to the step of inserting the tubular apparatus into the body.

42. The method of claim 40, further comprising the step of ultrasonically confirming the position of the tubular apparatus at or near the vocal folds prior to said step of advancing the tubular apparatus to a position at or near the patient's epiglottis.

43. The method of claim 40, wherein the step of ultrasonically imaging the location of the tubular apparatus within the body comprises the steps of:
directing an ultrasonic beam through the surface of the skin and into the leader balloon cuff; and
viewing the resulting image on an ultrasonic monitor.

44. The method of claim 43, wherein the ultrasonic beam is passed through the patient's middle cricothyroid ligament.

45. The method of claim 43, wherein the ultrasonic monitor is adapted to ultrasonically image fluid flow within the leader balloon cuff using Doppler imaging.

46. The method of claim 40, further comprising the steps of ventilating the patient using an external ventilating unit operatively coupled to the ventilation lumen.

47. A method of ultrasonically placing and monitoring an endotracheal tubular apparatus within a patient's airway, comprising the steps of:
providing at least one ultrasonic transducer on the anterior surface of the patient's neck;
providing an endotracheal tubular apparatus including a ventilation lumen, a main balloon cuff in fluid communication with a first external fluid source, and a leader balloon cuff in fluid communication with a second external fluid source;
inserting at least a portion of the tubular apparatus into the patient's oral or nasal cavity;
advancing the tubular apparatus to a first position at or near the patient's vocal folds;
inflating the leader balloon cuff with an acoustically transmissive material and ultrasonically confirm placement of the tubular apparatus at or near the vocal folds;
advancing the tubular apparatus to a second position at or near the patient's epiglottis;
inflating the leader balloon cuff with an acoustically transmissive material and ultrasonically confirm placement of the tubular apparatus at or near the epiglottis;
advancing the tubular apparatus to a third position within the trachea; and
inflating the leader balloon cuff with an acoustically transmissive material and ultrasonically confirm placement of the tubular apparatus within the trachea.

48. A method of ultrasonically placing and monitoring a tubular member within a patient's body, comprising the steps of:
providing at least one ultrasonic transducer located outside of the patient's body;
providing a tubular member having a proximal section, a distal section, and at least one inflation member operatively coupled to the distal section, said at least one inflation member being in fluid communication with a fluid source containing an acoustically transmissive material;
inserting the distal section of the tubular member into a hollow body cavity or conduit;
inflating said at least one inflatable member with the acoustically transmissive material; and
ultrasonically imaging the location of the tubular member within the body.
